(19) United States
(12) Patent Application Publication
(10) Pub. No.: US 2017/0224235 A1
(43) Pub. Date: Aug. 10, 2017

(54) MEASUREMENT APPARATUS AND MEASUREMENT METHOD

(71) Applicant: KYOCERA Corporation, Kyoto (JP)

(72) Inventor: Kinya SUGIMOTO, Okaya-shi, Nagano (JP)

(73) Assignee: KYOCERA Corporation, Kyoto (JP)

(21) Appl. No.: 15/503,170

(22) PCT Filed: Aug. 24, 2015
(86) PCT No.: PCT/JP2015/004230

§ 371 Date: Feb. 10, 2017

(30) Foreign Application Priority Data
Aug. 27, 2014 (JP) 2014-172943

(51) Int. Cl. A61B 5/026; A61B 5/00

(52) U.S. Cl. CPC A61B 5/0261; A61B 5/026; A61B 5/7275; A61B 5/6898; A61B 2560/0257

(57) ABSTRACT

A measurement apparatus for measuring biological information when a test site is in contact with a contact unit includes a biological sensor for acquiring a biometric output from the test site, a controller for measuring the biological information based on the biometric output, an arithmetic logic unit for calculating information on measurement accuracy of the biological information, and an information output unit for outputting the biological information and the information on the measurement accuracy.

### CROSS REFERENCE TO RELATED APPLICATION

This application claims priority to and the benefit of Japanese Patent Application No. 2014-172943 (filed on Aug. 27, 2014), the entire contents of which are incorporated herein by reference.

### TECHNICAL FIELD

This disclosure relates to a measurement apparatus and a measurement method.

### BACKGROUND

Conventionally, there is known a measurement apparatus for measuring biological information by acquiring a biological information output from a test site such as a subject's (user's) fingertip.

### SUMMARY

A measurement apparatus of the disclosure is a measurement apparatus for measuring biological information when a test site is in contact with a contact unit, the measurement apparatus includes:

a biological sensor for acquiring a biometric output from the test site;

a controller for measuring the biological information based on the biometric output;

an arithmetic logic unit for calculating information on measurement accuracy of the biological information; and

an information output unit for outputting the biological information and the information on the measurement accuracy.

Note that a method substantially corresponding to the measurement apparatus described above may also implement the disclosure and thus is included in the scope of the disclosure.

For example, a measurement method of the disclosure includes:

in measuring biological information when a test site is in contact with a contact unit,

a step in which a biological osensor acquires a biometric output from the test site;

a step in which a controller measures the biological information based on the biometric output;

a step in which an arithmetic logic unit calculates information on measurement accuracy of the biological information; and

a step in which an information output unit outputs the biological information and the information on the measurement accuracy.

### BRIEF DESCRIPTION OF THE DRAWINGS

In the accompanying drawings:

FIG. 1 is a functional block diagram illustrating a schematic configuration of a measurement apparatus according to one embodiment;

FIG. 2 is a diagram illustrating an example of a using state of the measurement apparatus;

FIG. 3 is a diagram illustrating an example of a display of a result of measurement and measurement accuracy;

FIG. 4 is a schematic diagram of a cross-section of a blood vessel;

FIGS. 5A and 5B are diagrams illustrating pressures applied to microelements within a blood vessel wall;

FIG. 6 is a diagram illustrating a relationship between a circumferential stress and its radial components;

FIGS. 7A and 7B are diagrams illustrating a displacement of a point within the blood vessel wall due to an influence of a pressure caused by a blood flow;

FIG. 8 is a schematic diagram illustrating a state in which the test site is in contact with the contact unit;

FIG. 9 is a flowchart illustrating an example of a measuring operation of the blood flow performed by a controller of FIG. 1;

FIG. 10 is a schematic diagram illustrating a blood vessel and a blood flow of a subject; and

FIGS. 11A and 11B are diagrams illustrating an example of a mobile phone having the measurement apparatus of FIG. 1 mounted thereon.

### DETAILED DESCRIPTION

A conventional blood flow measurement apparatus for measuring a blood flow as the biological information emits laser light to the fingertip and measures the blood flow based on scattered light from the blood flowing in the capillaries at the fingertip.

While measurement accuracy of the biological information varies depending on a condition of the capillaries in the test site, the condition of the capillaries varies each time of the measurement depending on a contact state of the test site in contact with the measurement apparatus. Although the capillaries are known to change their conditions due to aging (e.g., see Non-Patent Literature (NPL) 1: Shizuyo Okuyama, Junichi Ushiyama, Ken Muramatsu, Mitsuyoshi Murayama, and Reiko Sasaki, “Relationship of common carotid artery vascular diameter, intima-media thickness, and blood pressure in young people”, [online], [searched on Aug. 14, 2014] on the Internet <URL: http://koara.lib.keio.ac.jp/xoonips/moduleshoonips/download.php/AN00135710-00480 001-0001.pdf?file_id=52435>, NPL 2: Hirofumi Tanaka, Frank A. Dinenno, Kevin D. Monahan, Christopher A. DeSouza, and Douglas R. Seals, “Carotid artery wall hypertrophy with age is related to local systolic blood pressure in healthy men”, [online], [searched on Aug. 14, 2014], on the Internet <URL: http://www.researchgate.net/publication/12183281_Carotid_artery_wall_hypertrophy_w ith_age_is_related_to_local_systolic_blood_pressure_in_healthy_men>, and NPL 3: Daniel H. O′leary, Joseph F. Polak, Richard A. Kronmal, Teri A. Manolio, Gregory L. Burke, Sidney K. Wolfson, Jr., “Carotid-artery intima and media thickness as a risk factor for myocardial infarction and stroke in older adults”, [online], [searched on Aug. 14, 2014], on the Internet <URL: http://info-centre.jenage.de/assets/pdfs/library/oleary_et_al_NEJoM_1999.pdf>), inner diameters of the capillaries are considered as approximately 5 μm to 10 μm, and a mean value of the inner diameters is approximately 7 μm (e.g., see NPL 4: Eiichi Ueno “Blood Vessel (What is a model of the blood vessel?)”, [online], [searched on Aug. 14, 2014], on the Internet <URL: http://bio-omix.tmd.ac.jp/document/2007graduates/UenoE.pdf#search=′% E6%8C %87+%E6%AF%9B%E7% B4%B0%E8%A1%80%E7%AE%A1+%E5%A4%AA′>).

Since a conventional measurement apparatus cannot inform the subject of accuracy of a result of measurement, the subject cannot determine reliability of the result of the measurement.

Therefore, it could be helpful to provide a measurement apparatus and a measurement method which are capable of enhancing convenience.

Hereinafter, one embodiment of the disclosure will be described in detail with reference to the drawings.

FIG. 1 is a functional block diagram illustrating a schematic configuration of a measurement apparatus according to one embodiment. A measurement apparatus 10 includes a pressure detection unit 11, an atmospheric pressure measurement unit 12, a biological sensor 13, a contact unit 14, a controller 15, a notification unit 16, a memory 17, a display 18, an input interface 19, and an arithmetic logic unit 20.

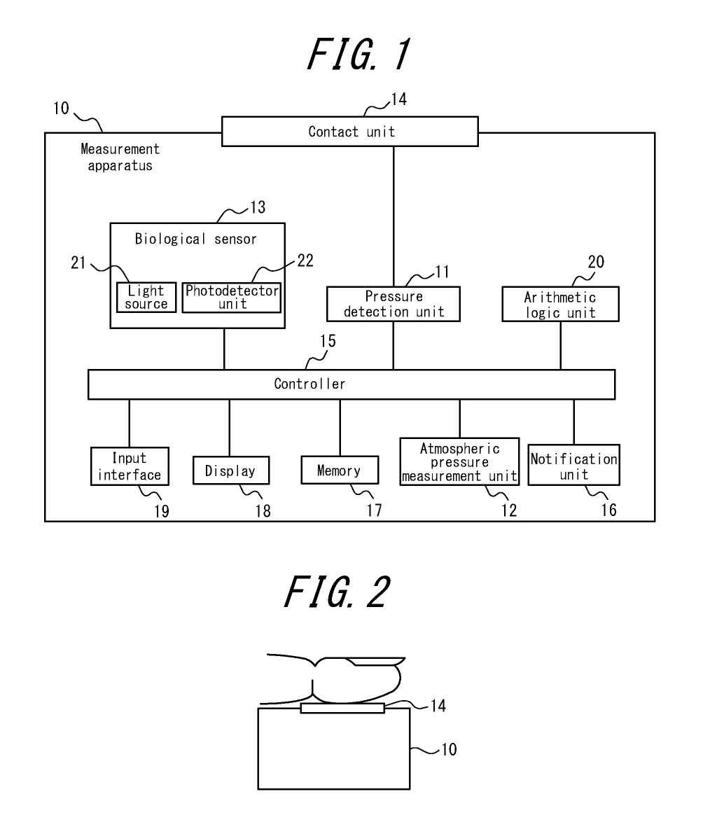

The measurement apparatus 10 measures biological information of a test site in contact with the contact unit 14. FIG. 2 is a diagram illustrating an example of a using condition of the measurement apparatus 10 in which a user is pressing the finger serving as the test site against the measurement apparatus 10. The measurement apparatus 10 measures the biological information when the finger is pressed against the contact unit 14 as illustrated in FIG. 2. Note that the biological information may be any biological information that may be measured by the biological sensor 13. In the present embodiment, the measurement apparatus 10 measures, by way of example, a blood flow of the subject serving as information on a flow of blood.

In FIG. 1, the pressure detection unit 11 detects a contact pressure applied to the contact unit 14. The pressure detection unit 11 may be, for example, a piezoelectric element. The pressure detection unit 11 is connected to the controller 15 and transmits a pressure signal representing a detected pressure to the controller 15. Therefore, when the test site is in contact with the contact unit 14, the pressure detection unit 11 detects the pressure applied to the contact unit 14 by the test site and transmits the pressure signal representing the detected pressure to the controller 15.

The atmospheric pressure measurement unit 12 measures an ambient atmospheric pressure surrounding the measurement apparatus 10. The atmospheric pressure measurement unit 12 may be a known barometer such as an aneroid barometer, a Bourdon-tube gauge, and an electric pressure meter. The atmospheric pressure measurement unit 12 is connected to the controller 15 and transmits a signal representing a measured atmospheric pressure to the controller 15. In the present embodiment, as described later, the atmospheric pressure measured by the atmospheric pressure measurement unit 12 is used for a calculation of measurement accuracy of the biological information.

The biological sensor 13 acquires a biometric output from the test site. When the measurement apparatus 10 measures the blood flow as described in the present embodiment, the biological sensor 13 includes a light source 21 and a photodetector unit 22.

The light source 21 emits laser light when controlled by the controller 15. For example, the light source 21 emits, as measurement light, the laser light in a wavelength which allows detection of a predetermined component contained in the blood and may be, for example, an LD (Laser Diode).

The photodetector unit 22 receives, as the biometric output, scattered light of the measurement light from the test site. The photodetector unit 22 may be, for example, a PD (Photodiode). The biological sensor 13 transmits a photoelectric conversion signal of the scattered light received by the photodetector unit 22 to the controller 15.

The contact unit 14 is a unit with which the test site such as a subject's finger is brought into contact by the subject for the measurement of the biological information. The contact unit 14 may be, for example, a plate-like member. The contact unit 14 is made of a transparent member to receive at least the measurement light from the light source 21 and the scattered light from the test site.

The controller 15 is a processor for controlling and managing the measurement apparatus 10 in its entirety including each functional block of the measurement apparatus 10. The controller 15 is a processor such as CPU (Central Processing Unit) for executing a program defining a control procedure stored in, for example, the memory 17 or an external storage medium.

The controller 15 controls emission of the laser light from the light source 21. For example, when the pressure detection unit 11 detects the contact pressure applied to the contact unit 14 after the subject sets the measurement apparatus 10 to be ready for the measurement of the biological information, the controller 15 controls the light source 21 such that the light source 21 emits the laser light. When the laser light is emitted, the biological sensor 13 starts acquiring the biometric output. After the biological sensor 13 starts acquiring the biometric output when the laser light is emitted, the controller 15 determines whether the biological sensor 13 has completed acquiring the biometric output. When the controller 15 determines that the acquisition of the biometric output is completed, the controller 15 stops an output of the laser light from the light source 21. The controller 15 may determine that the acquisition of the biometric output is completed when, for example, a predetermined time period has elapsed after the biological sensor 13 starts acquiring the biometric output. Alternatively, the controller 15 may determine that the acquisition of the biometric output is completed when, for example, the biological sensor 13 acquires the biometric output in a sufficient quantity for the measurement of the biological information. In this manner, the controller 15 controls the acquisition of the biometric output by the biological sensor 13.

The controller 15 acquires the pressure signal from the pressure detection unit 11. Then, the controller 15 determines whether the contact pressure applied to the contact unit 14 by the test site is equal to or higher than a predetermined pressure. The predetermined pressure may be, for example, a pressure which allows information on the measurement accuracy calculated based on a calculation algorithm used to derive the measurement accuracy described later to be functional and preliminarily stored in, for example, the memory 17.

When the controller 15 determines that the contact pressure applied to the contact unit 14 by the test site is equal to or higher than the predetermined pressure, the controller 15 controls the arithmetic logic unit 20 such that the arithmetic logic unit 20 calculates the measurement accuracy. That is, the arithmetic logic unit 20, when controlled by the controller 15, calculates the measurement accuracy of the biological information by using the calculation algorithm. The arithmetic logic unit 20 calculates the measurement accuracy of the biological information with respect to, for example, time. The arithmetic logic unit 20 may calculate a value representing the measurement accuracy throughout a time period of the measurement of the biological information. Note that a detailed description of the calculation algorithm will be presented infra. Also, although in the present embodiment the arithmetic logic unit 20 is a function unit independent of the controller 15, a function of the arithmetic logic unit 20 may be included in the controller 15. In this case, the controller 15 calculates the measurement accuracy.

When the controller 15 determines that the contact pressure applied to the contact unit 14 by the test site is lower than the predetermined pressure, the controller 15 controls the notification unit 16 such that the notification unit 16 issues notification of information on the contact pressure. The notification of the information on the contact pressure is, for example, notification to notify the subject of that the contact pressure is lower than the predetermined pressure. The notification of the information on the contact pressure may be an instruction to increase the contact pressure.

The notification unit 16 may perform the notification, for example, in a visual manner using an image, a character, and lighting, in an auditory manner using a voice and the like, or a combination thereof. The notification unit 16, in order to notify in the visual manner, displays, for example, the image or the character in a display device such as the display 18. The notification unit 16 may notify by, for example, turning on a light emitter such as an LED. The notification unit 16, in order to notify in the auditory manner, outputs, for example, an alarm sound, a voice guidance, and the like from a sound generating device such as a speaker. The notification issued by the notification unit 16 is not limited to the visual manner nor the auditory manner but may by any manner perceivable by the subject.

When the controller 15 determines that the biological sensor 13 has completed acquiring the biometric output, the controller 15 generates the biological information based on a biological information output (an output from the photodetector unit 22). Although in the measurement apparatus 10 of the present embodiment the controller 15 generates the biological information, a function unit independent of the controller 15 may generate the biological information.

Here, a blood flow measuring technology using a Doppler shift employed by the controller 15 will be described. The controller 15, in order to measure the blood flow, controls the light source 21 such that the light source 21 emits the laser light into tissues (the test site) of a living body such that the photodetector unit 22 receives the scattered light from the inside of the tissues of the living body. Then, the controller 15 calculates the blood flow based on an output associated with the scattered light received.

Inside the tissues of the living body, the scattered light scattered by the blood cells which are moving are subjected to a frequency shift (the Doppler shift) due to Doppler effect which increases in proportion to a moving speed of the blood cells in the blood. The controller 15 detects an Unari-signal (also referred to as a beat signal) generated by optical interference between the scattered light from still tissues and the scattered light from the blood cells which are moving. The beat signal indicates intensity represented by a time function. Then, the controller 15, from the beat signal, generates a power spectrum in which power is represented by a frequency function. In the power spectrum of the beat signal, the Doppler shift frequency increases in proportion to the moving speed of the blood cells, and the power corresponds to an amount of the blood cells. Then, the controller 15 calculates the blood flow by multiplying the power spectrum of the beat signal by the frequency and integrating.

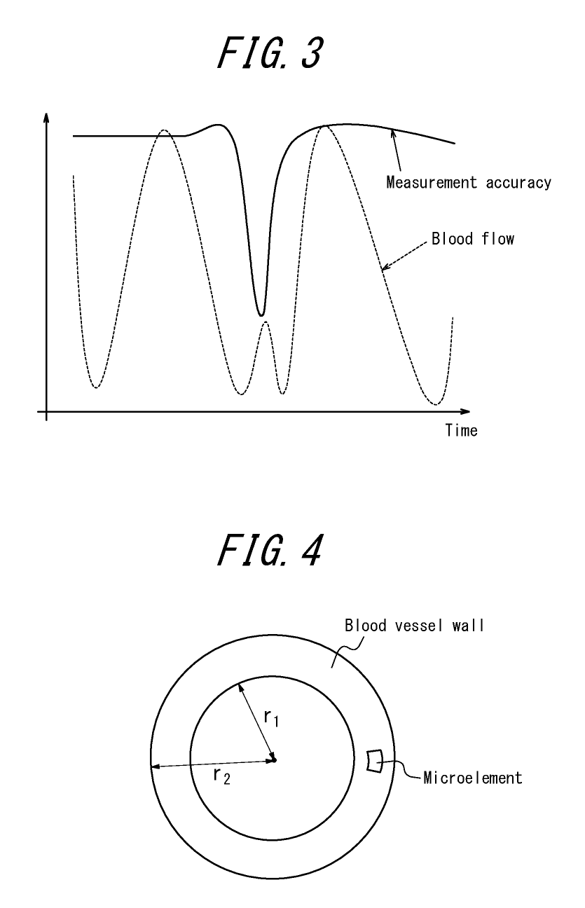

The controller 15 displays measured biological information in the display 18. When the controller 15 calculates the measurement accuracy, the controller 15 displays calculated measurement accuracy together with the biological information in the display 18. FIG. 3 is a diagram illustrating an example of a display of a result of the measurement and the measurement accuracy. The controller 15, as illustrated in FIG. 3, for example, displays a change in the blood flow and a change in the measurement accuracy with respect to time as the result of the measurement. In the example illustrated in FIG. 3, the measurement accuracy is represented by a solid line, and the blood flow is represented by a dotted line.

When the measurement accuracy is not calculated due to the contact pressure lower than the predetermined pressure, the controller 15 may display, together with the biological information, an indication that the measurement accuracy of the result of the measurement being displayed is very poor.

Of three maximum values of the blood flow illustrated in FIG. 3, a maximum value in a center is lower than the other maximum values. At a time point when the maximum value in the center is measured, the measurement accuracy is temporarily low. This is because the contact pressure is appropriate for the measurement of the blood flow before and after the maximum value in the center is measured, while the contact pressure is temporality too high, or too low, at the time point when the maximum value in the center is measured and thus the blood flow cannot be measured appropriately.

The memory 17 may be a memory such as a semiconductor memory and a magnetic memory and stores various information and programs for operating the measurement apparatus 10 and also functions as a work memory. The memory 17 stores, for example, the blood flow measured by the measurement apparatus 10 in association with the measurement accuracy.

The memory 17 storage various information used by the arithmetic logic unit 20 to calculate the measurement accuracy. For example, the memory 17 stores the predetermined pressure used as a criterion as to whether to calculate the measurement accuracy using the calculation algorithm. The memory 17 also stores information on the subject necessary for the calculation of the measurement accuracy by the arithmetic logic unit 20. The information on the subject includes, for example, subject's gender, age, height, and normal blood pressure and is input by the subject with, for example, the input interface 19. The normal blood pressure is, for example, blood pressure preliminarily measured by the subject with a known sphygmomanometer.

The memory 17 further stores the calculation algorithm used by the arithmetic logic unit 20 for the calculation of the measurement accuracy and information (data) necessary for the controller 15 in using the calculation algorithm. The information necessary for the controller 15 in using the calculation algorithm is, for example, data indicating a relationship between a standard inner diameter and a standard outer diameter of the blood vessel for ages. Changes in the blood vessel with aging is described in, for example, NPLs 1 to 3. The controller 15, based on the age input by the subject, refers to the data stored in the memory 17 and determines the inner and outer diameters of the blood vessel of the subject. Note that in the present embodiment the information stored in the memory 17 is not limited to the data indicating the relationship between the standard inner and outer diameters of the blood vessel and age. Examples of other information stored in the memory 17 will be presented below in a description of the calculation algorithm executed by the arithmetic logic unit 20. The arithmetic logic unit 20 executes the calculation algorithm referring to the data stored in the memory 17 and calculates the measurement accuracy of the biological information.

The display 18 is a display device such as a liquid crystal display, an organic EL display, and an inorganic EL display. The display 18 displays, for example, the result of the measurement of the biological information conducted by the measurement apparatus 10.

The input interface 19 receives an input operation from the subject and is, for example, an operation button (an operation key). The input interface 19 may be a touch panel and displayed in a portion of the display 18 so as to receive a touch input operation from the subject. The subject may, for example, run a dedicated application for the measurement of the biological information by operating the input interface 19 of an electronic apparatus such as the mobile phone having the measurement apparatus 10 mounted thereon.

Next, the calculation of the measurement accuracy performed by the arithmetic logic unit 20 will be described. The arithmetic logic unit 20 calculates the measurement accuracy based on, for example, a change rate of an inner diameter of the blood vessel. In particular, the arithmetic logic unit 20 calculates the measurement accuracy from Formula (1).

[Formula 1]

r_{I}+(uₚ+uₛ)/r₁ (1)

In the Formula (1), the r_{I} represents a normal inner diameter of the blood vessel. The normal inner diameter r_{I} of the blood vessel is determined by the controller 15 based on, for example, the gender and age input by the subject, by referring to the data about the standard inner diameter of the blood vessel of corresponding gender and age stored in the memory 17. Note that the inner diameter r_{I} of the blood vessel is approximately 5 μm to 10 μm, and a mean value of the inner diameters is approximately 7 μm. The u_{P} represents a displacement of the inner diameter of the blood vessel due to an influence of the blood flow within the blood vessel, and the u_{S} represents a displacement of the inner diameter of the blood vessel due to an influence of the contact pressure applied by the test site in contact with the contact unit 14. The displacement u_{P} of the inner diameter of the blood vessel due to the influence of the blood flow and the displacement u_{S} of the inner diameter of the blood vessel due to the influence of the contact pressure are calculated by the arithmetic logic unit 20 by using a predetermined calculation algorithm.

The calculation algorithm of the present embodiment, assuming that the blood vessel in the test site is in an ideal cylindrical shape having elasticity, presumes the displacement of the inner diameter of the blood vessel caused by the pressure applied by a user and calculates the change in the inner diameter of the blood vessel. Note that the calculation algorithm is not limited to one disclosed herein but may be any algorithm capable of allowing a presumption of the displacement of the inner diameter of the blood vessel.

Here, the inner diameter r_{I} of the blood vessel changes due to the influence of the blood flow within the blood vessel and an externally applied pressure (the contact pressure in the present embodiment). That is, the blood vessel receives a pressure for increasing the inner diameter r_{I} of the blood vessel from the blood flow within the blood vessel and, also, a pressure for reducing the inner diameter r_{I} due to the contact pressure. Note that the calculation algorithm used in the present embodiment assumes that a stress is generated in radial and circumferential directions of the blood vessel but not in a longitudinal direction of the blood vessel. In the following description with reference to FIG. 4 to FIG. 8, microelements displayed are enlarged, for convenience of description. Also, an arrow associated with the microelements indicates a direction of each element. Therefore, a start point of the arrow in the figure does not necessarily correspond to a point of action of the element indicated by the arrow.

As to the change in the inner diameter r_{I} of the blood vessel, the change caused by the blood flow will be described first. FIG. 4 is a schematic diagram of a cross-section of the blood vessel. A pressure applied by the blood flow in the radial direction of the blood vessel to the microelements in a fan-shaped area within a blood vessel wall as illustrated in FIG. 4 will be described. Here, a coordinate having an origin representing the center of the blood vessel and r and t respectively representing the radial direction and the circumferential direction is used. An X-axis is an axis in the same direction as the radial direction r of the microelements, and a Y-axis (an axis in a tangential direction) is an axis orthogonal to the X-axis.

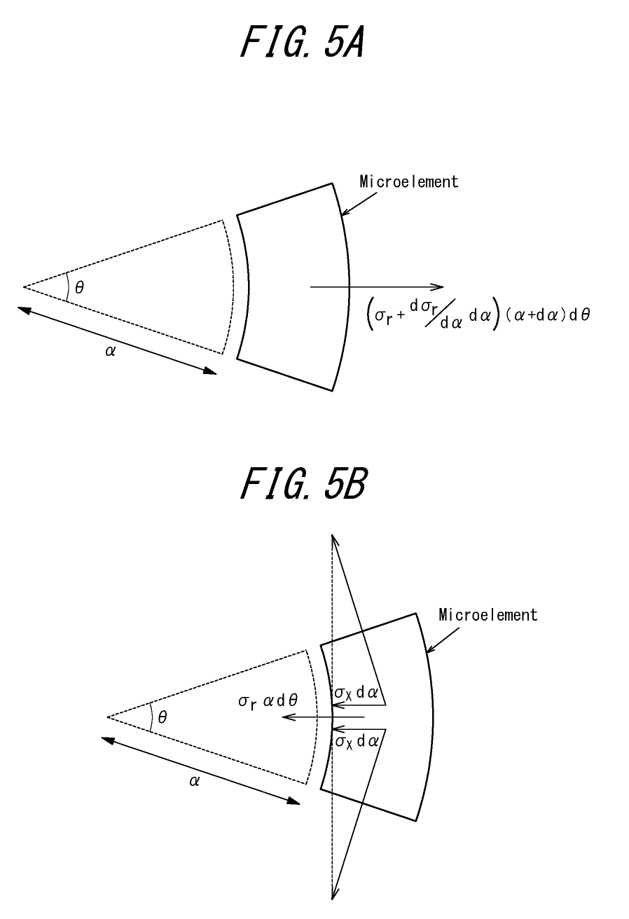

FIGS. 5A and 5B are diagrams illustrating pressures applied to the microelements within the blood vessel wall. Here, a represents a distance between the center of the blood vessel and the microelements (i.e., r=α), and θ represents a central angle formed by the fan-shaped microelements. Also, σᵣ represents a pressure in the radial direction (an r-direction) applied to the microelements within the blood vessel wall. At this time, a pressure applied in an outer diameter direction of the microelements illustrated in FIG. 5A is expressed by Formula (2).

[Formula 2]

(σₜ+dσₜ/dα^{dα})(α+dα)dθ (2)

On the other hand, a pressure applied in an inner diameter direction of the microelements illustrated in FIG. 5B is expressed by a sum of a radial (the r-direction) stress applied to the microelements and elements in the radial direction of a circumferential (a t-direction) stress σₜ. Therefore, provided that σ_{X} represents a radial direction element of the circumferential (the t-direction) stress σₜ, a pressure applied in the inner diameter direction of the microelements is expressed by Formula (3).

[Formula 3]

σᵣαdθ+2σ_{X}dα (3)

In a stationary state, since the pressure applied in the outer diameter direction of microelements and the pressure applied in the inner diameter direction of the microelements are balanced out, Formula (4) is satisfied based on the Formula (2) and the Formula (3).

[Formula 4]

(σₜ+dσₜ/dα^{dα})(α+dα)dθ−σₜαdθ-31 2σₓdα=0 (4)

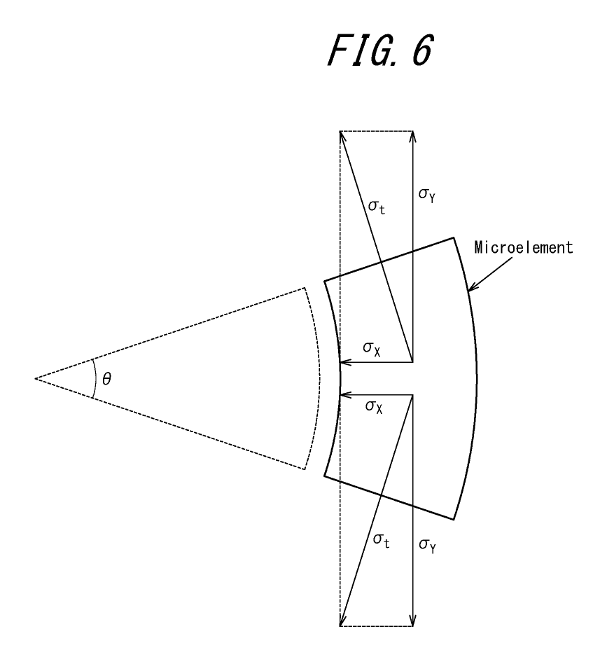

Here, as illustrated in FIG. 6, since the radial direction element σ_{X} of the circumferential stress σₜ is an orthogonal projection on the radial direction (the X-axis) of the circumferential stress σₜ, the radial direction element σ_{X} of the circumferential stress σₜ is expressed by Formula (5).

[Formula 5]

σₓ=σₜsin(dθ/2)≅σₜ/2^{dθ} (5)

By substituting Formula (5) in Formula (4) and rearranging, Formula (6) is obtained.

[Formula 6]

dσₜ/dασₜ+dσₜ/dαdα−σₜ=0 (6)

By ignoring small terms in the Formula (6), Formula (7) is obtained.

[Formula 7]

dσₜ/dα+σᵣ−σₜ=0 (7)

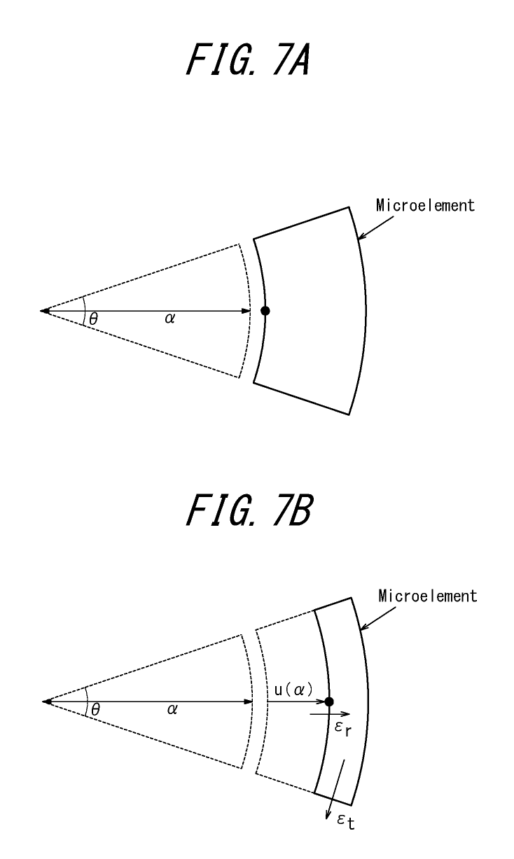

Here, the displacement due to the influence of the pressure caused by the blood flow at one point within the blood vessel wall distant from the center of the blood vessel by a (i.e., r=a) is considered. FIGS. 7A and 7B are diagrams illustrating the displacement of the one point within the blood vessel wall due to the influence of the pressure caused by the blood flow. Here, since the blood vessel is assumed to have the ideal cylindrical shape and elasticity, a circumference of a radius a of the blood vessel wall uniformly stretches and contracts. When u(α) represents a radial displacement of the one point within the blood vessel from a state in which there is no influence of the pressure caused by the blood vessel as illustrated in FIG. 7A to a state in which there is the influence of the pressure caused by the blood vessel as illustrated in FIG. 7B, a displacement of the radial α+dα is expressed by Formula (8).

[Formula 8]

u(α+dα)=u(α)+du/dαdα (8)

Here, based on a definition formula of strain: ε=ΔL/L, a radial strain εᵣ at the one point within the blood vessel wall is expressed by Formula (9).

[Formula 9]

ε=u(α+dα)/dα=du/dα (9)

Similarly, a circumferential strain εₜ at the one point within the blood vessel wall is expressed by Formula (10).

[Formula 10]

ε₁=2π(α+u)-31 2πα/2πα=u/α (10)

Here, since it is assumed that no stress is generated in the longitudinal direction of the blood vessel, in a general x-y plane coordinate a relational expression of two-dimensional stress expressed by Formula (11) and Formula (12) are satisfied. Note that E represents a longitudinal elasticity modulus inherent to the vessel, and v represents a Poisson's ratio. The longitudinal elasticity modulus E and the Poisson's ratio v are determined by the arithmetic logic unit 20 based on the information on the subject by referring to the data stored in the memory 17.

[Formula 11]

σₓ=E/1−v²(εₓ+vε_{y}) (11)

[Formula 12]

σ_{y}=E/1−v²(ε_{y}+vεₓ) (12)

By substituting the Formula (9) and the Formula (10) respectively in the Formula (11) and the Formula (12) and also substituting r for x and t for y, Formula (13) and Formula (14) are obtained.

[Formula 13]

σᵣ=E/1−v²(εᵣ+vεₜ)=E/1−v²(du/dα+v^{u}/α) (13)

[Formula 14]

σᵣ=E/1−v²(εₜ+vεᵣ)=E/1−v²(u/α+v^{du}/dα) (14)

By substituting the Formula (13) and the Formula (14) in the Formula (7) and calculating, Formula (15) is obtained.

[Formula 15]

d²u/da²+1/αdu/dα−u/α²=0 (15)

By transforming the Formula (15), a differential formula expressed by Formula (16) is obtained.

[Formula 16]

d²u/da²+d/dα(u/α)=0 (16)

By solving the differential formula expressed by Formula (16), Formula (17) is obtained including integration constants c₁ and c₂.

[Formula 17]

U=c₁αc₂/α (17)

By differentiating both sides of the Formula (17), Formula (18) is obtained.

[Formula 18]

du/dα=c₁−c₂/α² (18)

By substituting the u obtained from the formula (17) and the dᵤ/dα obtained from the Formula (18) in the formula (13) and calculating, a general solution to σᵣ is obtained.

[Formula 19]

σᵣ=E/1−v²du/dα+v^{u}/α)=E/1−v²((1+v)c₁−(1−v)c₂I/α₂₎ (19)

Here, the integration constants c₁ and c₂ are obtained from a boundary condition. As illustrated in FIG. 4, r₁ represents the inner radius of the blood vessel, and r₂ represents an outer radius of the blood vessel. When P represents a pressure applied to an inner surface of the blood vessel wall from the blood vessel wall, σᵣ=−P is satisfied, provided that α=r₁. By substituting this condition in the Formula (19), Formula (20) is obtained.

[Formula 20]

E/1−v²((1+v)c₁−(1−v)c₂1/r₁²)=−P (20)

Here, the P is obtained by adding a hydrostatic pressure to an internal pressure of the blood vessel caused by the blood flow. When P₀ represents a barometric pressure, ρ represents blood density, g represents gravitational acceleration, and h represents a displacement of a height of the test site from the heart, the P is expressed by Formula (21). The barometric pressure P₀ is the ambient atmospheric pressure surrounding the measurement apparatus 10 measured by the pressure measurement unit 12. The blood density ρ is determined by the arithmetic logic unit 20 based on the gender input by the subject by referring to, for example, the blood density of each gender stored in the memory 17. The displacement h of the height of the test site from the heart is determined by the arithmetic logic unit 20 based on the height input by the subject by referring to, for example, data of a relationship between a standard height of the heart for each height and a height of a position of the test site in a measuring posture of the biological information stored in the memory 17.

[Formula 21]

P=P₀+ρgh (21)

Next, as to an outer surface of the blood vessel wall, since there is no pressure applied to the outer surface of the vessel wall, σᵣ=0 is satisfied, provided that α=r₂. By substituting this condition in the Formula (19), Formula (22) is obtained.

[Formula 22]

E/1−v²((1+v)c₁−(1−v)c₂1/r₂²)=0 (22)

By solving the Formula (20) and the Formula (22), the integral constants c₁ and c₂ are obtained. The integration constants c₁ and c₂ are respectively expressed by Formula (23) and Formula (24). Note that n=r₁/r₂ is satisfied.

[Formula 23]

c₁=(1−v/e)(n²P/1−n²) (23)

By substituting the integration constants c₁ and c₂ respectively calculated from the Formula (23) and the Formula (24) in the Formula (19), a solution to the radial pressure σᵣ₁ within the blood vessel wall is obtained. The solution to the pressure σᵣ₁ is expressed by Formula (25).

[Formula 25]

σᵣ₁=P/1−n²(n²−r₁²/α²) (25)

Further, by substituting the integration constants c₁ and c₂ respectively calculated from the Formula (23) and the Formula (24) in the Formula (17), a solution to the displacement u₁ of the blood vessel wall due to the influence of the blood flow is obtained. The solution to the displacement u₁ is expressed by Formula (26).

[Formula 26]

u₁=P/E(1−n²)((1−v)n²α+(1+v)r₁²/α (26)

Here, the displacement u_{P} of the inner diameter of the blood vessel due to the influence of the blood flow is satisfied, provided that α=r₁. Therefore, when the displacement u_{P} substitutes in the Formula (26), the displacement u_{P} of the inner diameter of the blood vessel due to the influence of the blood flow is obtained. The displacement u_{P} of the inner diameter of the blood vessel due to the influence of the blood flow is expressed by Formula (27).

[Formula 27]

u₁=P/E(1−n²)(r₁(((1−v)n²+(1+v))) (27)

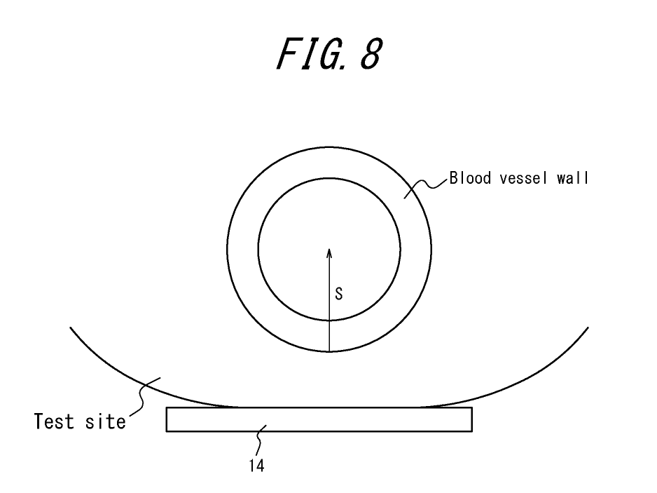

Next, as to the change in the inner diameter r_{I} of the blood vessel, the change due to the influence of the contact pressure will be described. FIG. 8 is a schematic diagram illustrating a state in which the test site is in contact with the contact unit 14, that is, a state in which the subject is measuring the biological information. As illustrated in FIG. 8, in accordance with the contact pressure of the test site in contact with the contact unit 14, the blood vessel receives the pressure from the contact unit 14 applied by the test site. This pressure causes the blood vessel to contract. The displacement u of the microelements in the blood vessel wall caused by the contraction of the blood vessel may be calculated similarly from the Formula (2) to the Formula (16) and expressed by the Formula (17).

Next, integration constants when the inner diameter r_{I} of the blood vessel changes due to the influence of the contact pressure will be obtained. Here, to distinguish from the change due to the influence of the blood flow as described above, the integration constants c₁ and c₂ in the Formula (17) will be respectively replaced with integration constants c₃ and c₄. Since the inner surface of the blood vessel wall is not affected by the contact pressure, σᵣ=0 is satisfied, provided that α=r₁. By substituting this condition in the Formula (19), Formula (28) is obtained.

[Formula 28]

E/1−v²((1+v)c₃−(1−v)c₄1/r₁²)=0 (28)

Further, as to the outer surface of the blood vessel wall, when S represents the pressure applied to the outer surface of the blood vessel wall from an outside of the blood vessel, σᵣ=−S is satisfied, provided that α=r₂. By substituting this condition in the Formula (19), Formula (29) is obtained.

[Formula 29]

E/1−v²((1+v)c₃−(1−v)c₄1/r₁²)=−S (29)

By solving the Formula (28) and Formula (29), the integral constants c₃ and c₄ are obtained. The integration constants c₃ and c₄ are respectively expressed by Formula (30) and Formula (31).

[Formula 30]

c₃=(1−v/E)(−S/1−n²) (30)

[Formula 31]

c₄=(1+v/E)(−r₁²S/1−m²) (31)

By substituting the integration constants c₃ and c₄ respectively calculated from the Formula (30) and the Formula (31) in the Formula (19), a solution to the radial pressure σᵣ₂ within the blood vessel wall due to the influence of the contact pressure is obtained. The solution to the radial pressure σᵣ₂ is expressed by Formula (32).

[Formula 32]

σᵣ₂=−S/1−n²(1−r₁²/a²) (32)

Further, by substituting the integration constants c₃ and c₄ respectively calculated from the Formula (30) and the Formula (31) in the Formula (17), a solution to the displacement u₂ of the blood vessel wall due to the influence of the contact pressure is obtained. The solution to the displacement u₂ is expressed by Formula (33).

[Formula 33]

u₂=−S/E(1−n²) ((1−v)Δ+(1+v)r₁²/a) (33)

Here, the displacement u_{S} of the inner diameter of the blood vessel due to the influence of the contact pressure is satisfied, provided that α=r₁. Therefore, by substituting this in the Formula (33), the displacement u_{S} of the inner diameter of the blood vessel due to the influence of the contact pressure is obtained. The displacement u_{S} of the inner diameter of the blood vessel due to the influence of the contact pressure is expressed by Formula (34).

[Formula 34]

u_{S}=−2r₁S/E(1−n²) (34)

Since the inner diameter r_{I} of the blood vessel changes due to the influence of the blood flow within the blood vessel and the contact pressure, the change in the inner diameter r_{I} is expressed by u_{P}+u_{S}. The arithmetic logic unit 20 calculates the measurement accuracy by calculating the Formula (1) using the displacement of the inner diameter thus obtained.

Note that the arithmetic logic unit 20 may determine 1 as a maximum value of the measurement accuracy expressed by the Formula (1). That is, when in the Formula (1) a result of u_{P}+u_{S} is a positive number, a calculation result of the Formula (1) is greater than 1, in which case the arithmetic logic unit 20 may determine the measurement accuracy as 1. Here, referring to the Formula (27) and the Formula (34) respectively expressing the u_{P} and the u_{S}, a sign of the u_{P} is positive, and a sign of the u_{S} is negative. That is, the result of u_{P}+u_{S} becomes positive when an absolute value of the u_{P} is larger than an absolute value of the u_{S}. Therefore, the arithmetic logic unit 20 may determine the measurement accuracy as 1 when the absolute value of the u_{P} is larger than the absolute value of the u_{S}.

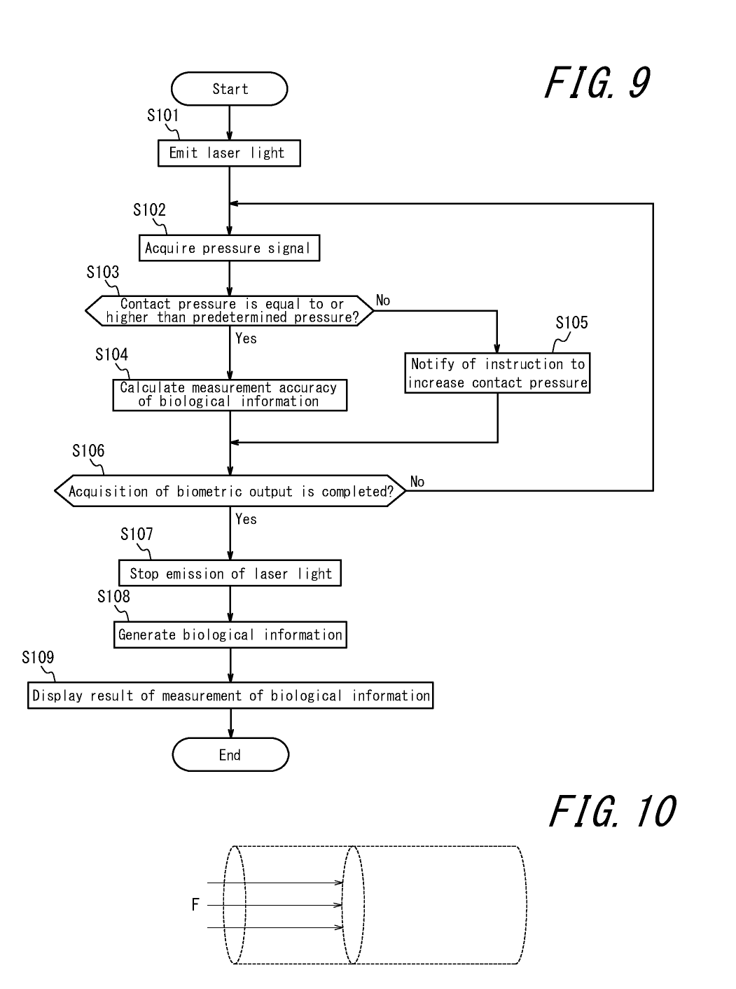

Next, an example of a measurement process of the blood flow performed by the controller 15 will be described with reference to a flowchart illustrated in FIG. 9. When the subject sets the measurement apparatus 10 to be ready for the measurement of the biological information by operating the measurement apparatus 10, the controller 15 starts a flow of FIG. 10.

When the pressure detection unit 11 detects the contact pressure applied to the contact unit 14, the controller 15 controls the light source 21 such that the light source 21 emits the laser light (step S101). When the laser light is emitted, the controller 15 controls the biological sensor 13 such that the biological sensor 13 starts acquiring the biometric output.

Next, the controller 15 acquires the pressure signal indicative of the information on the contact pressure to the contact unit 14 detected by the pressure detection unit 11 (step S102).

The controller 15, based on the pressure signal acquired, determines whether the contact pressure to the contact unit 14 is equal to or higher than the predetermined pressure stored in the memory 17 (step S103).

When determining that the contact pressure is equal to or higher than the predetermined pressure (Yes at step S103), the controller 15 controls the arithmetic logic unit 20 such that the arithmetic logic unit 20 executes the calculation algorithm described above and calculates the measurement accuracy derived from the Formula (1) (step S104). At this time, the controller 15 stores the measurement accuracy calculated by the arithmetic logic unit 20 in the memory 17. Then, the controller 15 proceeds to step S106.

On the other hand, when determining that the contact pressure is lower than the predetermined pressure (No at step S103), the controller 15 controls the notification unit 16 such that the notification unit 16 issues the instruction to increase the contact pressure (step S105). Then, the controller 15 proceeds to step S106.

After step S104 or step S105, the controller 15 determines whether the biological sensor 13 has completed acquiring the biometric output (step S106).

When the controller 15 determines that the acquisition of the biometric output is not completed (No at step S106), the controller 15 returns to step S102 while maintaining the acquisition of the biometric output.

When the controller 15 determines that the acquisition of the biometric output is completed (Yes at step S106), the controller 15 controls the light source 21 such that the light source 21 stops emitting the laser light (step S107).

The controller 15 generates the biological information based on the biometric output acquired (step S108). At this time, the controller 15 may store the biological information thus generated in the memory 17.

Then, the controller 15 displays the result of the measurement of the biological information in the display 18 (step S109). When at step S104 the arithmetic logic unit 20 calculates the measurement accuracy of the biological information (Yes at step S103), the controller 15 controls to display the result of the measurement of the biological information together with the measurement accuracy calculated by the arithmetic logic unit 20. When the arithmetic logic unit 20 does not calculate the measurement accuracy of the biological information (No at step S103), the controller 15 may control to display the indication that the measurement accuracy is very poor. For example, the controller 15 may control to display a value “0” as information on the measurement accuracy.

As described above, the measurement apparatus 10 of the present embodiment displays the measurement accuracy of the biological information together with the result of the measurement of the biological information in the display 18, thereby notifying the subject. Therefore, the subject may check the measurement accuracy of the result of the measurement displayed and determine whether the result of the measurement is highly reliable. Accordingly, the measurement apparatus 10 enhances convenience. Also, when the contact pressure is equal to or higher than the predetermined pressure, the controller 15 controls the arithmetic logic unit 20 such that the arithmetic logic unit 20 calculates the information on the measurement accuracy, while controlling the arithmetic logic unit 20, when the contact pressure is lower than the predetermined pressure, such that the arithmetic logic unit 20 does not calculate the information on the measurement accuracy. That is, the controller 15, when the measurement accuracy is functional, controls such that the information on the measurement accuracy calculated by using the calculation algorithm is calculated. Therefore, the reliability of the measurement accuracy may be maintained at least at a certain level. Further, when the contact pressure is lower than the predetermined pressure, the controller 15 controls the notification unit 16 such that the notification unit 16 issues the instruction to increase the contact pressure. Therefore, the subject may increase the pressure, and the contact pressure is more likely to be increased at least to the predetermined pressure. Consequently, the measurement apparatus 10 is more likely to display the measurement accuracy. Since the measurement apparatus 10 measures the biological information regardless of whether the contact pressure is equal to or higher than the predetermined pressure, the measurement apparatus 10 may reduce a nuisance to the subject which would otherwise be caused by a measurement apparatus which requires the contact pressure to meet a predetermined pressure for the start of the measurement.

The above embodiment should not be construed to limit the disclosure but may be modified or changed in a variety of manners. For example, functions and the like included in each constituent and step may be rearranged without logical inconsistency, so as to combine a plurality of constituents or steps together or to separate them.

For example, although in the above embodiment the biological information and the information on the measurement accuracy are displayed in the display 18, a method of notifying the subject of the biological information and the information on the measurement accuracy is not limited to the display 18. The measurement apparatus 10 simply needs to include any information output unit capable of notifying the subject of the biological information and the information on the measurement accuracy. Such an information output unit, similarly to the notification unit 16 in the above embodiment, may output the information in any manner including the visual manner, the auditory manner, and other manners perceivable by the subject.

Although in the above embodiment the arithmetic logic unit 20 calculates the measurement accuracy from the Formula (1), the measurement accuracy may be calculated from any other appropriate formulas. For example, the arithmetic logic unit 20 may calculate the measurement accuracy from Formula (35).

$$\begin{matrix}[ Formula 35 ] & \\ \sqrt[4]{F} + ( {u}_{p} + {u}_{s} ) / \sqrt[4]{F} & ( 35 )\end{matrix}$$

In the Formula (35), the F represents an amount of blood passing through a given cross-section of the blood vessel per unit time. FIG. 10 is a schematic view illustrating the blood vessel and the blood flow of the subject. The amount F of the blood passing through the cross-section per unit time is expressed by Formula (36) by using a pulse pressure ΔP and vascular resistance R. Note that the pulse pressure ΔP is a difference between a systolic blood pressure (a maximum blood pressure) and a diastolic blood pressure (a minimum blood pressure).

[Formula 36]

F=ΔP/R (36)

In the Formula (36), the vascular resistance R is expressed by Formula (37) by using blood viscosity V, a length of the blood vessel L, and the inner diameter r_{I} of the blood vessel. The blood viscosity V is determined by the arithmetic logic unit 20 based on, for example, the information on the subject by referring to the data stored in the memory 17.

[Formula 37]

R=(VL/r²)(8/90 ) (37)

In another example, the arithmetic logic unit 20 may calculate the measurement accuracy from, for example, Formula (38).

$$\begin{matrix}[ Formula 38 ] & \\ \overline{r + ( {u}_{r} + u ) \text{/} r} & ( 38 )\end{matrix}$$

The Formula (38) expresses a mean change rate obtained by sampling the Formula (1) in unit time. When the arithmetic logic unit 20 calculates the measurement accuracy from the Formula (38), the arithmetic logic unit 20 calculates, as the measurement accuracy, a mean value in a time period through which the biometric output is acquired. In this case, therefore, the controller 15 displays a value indicative of the measurement accuracy, rather than the graph illustrated in FIG. 3, in the display 18.

In yet another example, the arithmetic logic unit 20 determines whether each sample of the biometric output satisfies Formula (39). Here, Tᵤₚₚₑᵣ and T_{lower} are predetermined thresholds satisfying Tᵤₚₚₑᵣ<T_{lower}.

$$\begin{matrix}[ Formula 39 ] & \\ {T}_{lower} < r + ( {u}_{p} + {u}_{s} ) \text{/} r < {T}_{upper} & ( 39 )\end{matrix}$$

The arithmetic logic unit 20 may count, out of all samples, samples which satisfy the Formula (39) and determine, as the measurement accuracy, a ratio of the samples satisfying the Formula (39) to all samples.

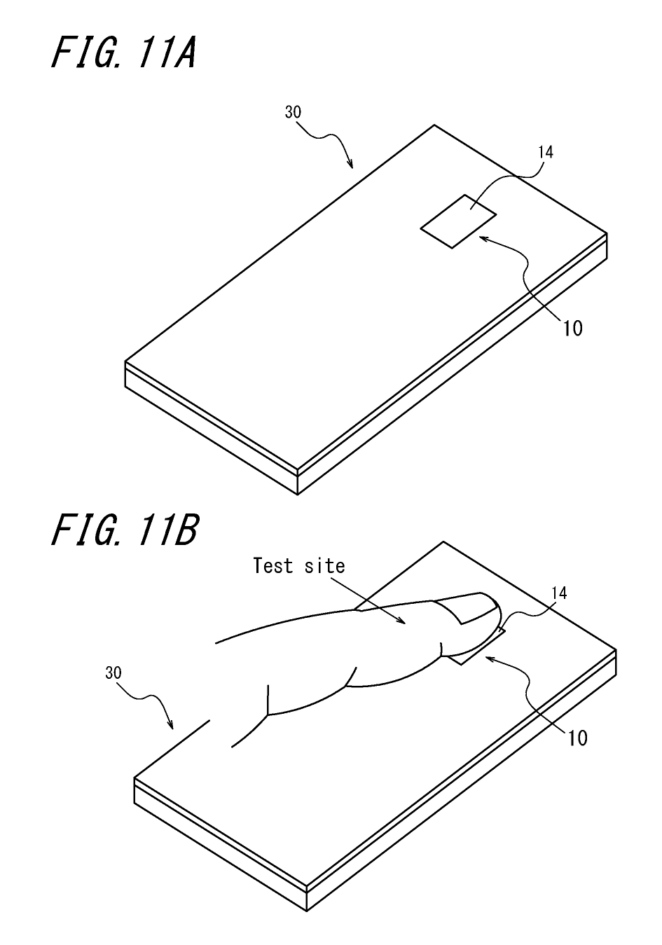

The measurement apparatus 10 according to the above embodiment may be mounted on various electronic apparatuses. FIGS. 11A and 11B are diagrams illustrating an example of a mobile phone having the measurement apparatus 10 of FIG. 1 mounted thereon. As illustrated in FIG. 11A, a mobile phone 30 has the measurement apparatus 10 mounted on a rear side thereof.

FIG. 11B is a diagram illustrating an example in which the subject is measuring the biological information with the mobile phone 30 having the measurement apparatus 10 mounted thereon. The subject measures the biological information with the measurement apparatus 10 by contacting the contact unit 14 of the measurement apparatus 10 with a finger.

When the measurement apparatus 10 is mounted on the electronic apparatus as illustrated in FIG. 11, a function of each function unit of the measurement apparatus 10 illustrated in FIG. 1 may be implemented by each function unit of the electronic apparatus. For example, the controller 15 may use a notification unit of the mobile phone 30 as the notification unit 16.

Arrangement of the measurement apparatus 10 in the mobile phone 30 is not limited to one illustrated in FIGS. 11A and 11B. The measurement apparatus 10 may be arranged, for example, in another position on a rear surface of the mobile phone 30, or on a front surface or a lateral side of the mobile phone 30.

The electronic apparatus having the measurement apparatus 10 mounted thereon is not limited to the mobile phone 30. For example, the measurement apparatus 10 may be mounted on a variety of electronic apparatuses including a portable music player, a laptop computer, a watch, a tablet computer, and a game machine.

Although in the above embodiment the controller 15 of the measurement apparatus 10 generates the biological information based on the output of the photodetector unit 22, the generation of the biological information does not need to be performed by the controller 15 of the measurement apparatus 10. For example, a server apparatus connected to the measurement apparatus 10 via a network configured with a wired connection, a wireless connection, or a combination thereof may include a function unit corresponding to the controller 15 and the arithmetic logic unit 20 and generates the biological information and calculates the measurement accuracy. In this case, the measurement apparatus 10 acquires the biological information output from the biological sensor 13 and transmits the biological information to the server apparatus via a communication unit separately provided. Then, the server apparatus generates the biological information based on the biological information output and calculates the measurement accuracy of the biological information by using the calculation algorithm. Subsequently, the server apparatus transmits the biological information generated and the measurement accuracy calculated to the measurement apparatus 10. The subject may view a result of the measurement when the biological information received by the measurement apparatus 10 is displayed together with the measurement accuracy in the display 18. When the server apparatus generates the biological information as described above, downsizing of the measurement apparatus 10 may be achieved, as compared to the measurement apparatus 10 including all function units illustrated in FIG. 1.

1. A measurement apparatus for measuring biological information when a test site is in contact with a contact unit, the measurement apparatus compri sing:
a biological sensor for acquiring a biometric output from the test site;
a controller for measuring the biological information based on the biometric output;
an arithmetic logic unit for calculating information on measurement accuracy of the biological information; and
an information output unit for outputting the biological information and the information on the measurement accuracy.

2. The measurement apparatus according to claim 1, further comprising:
an atmospheric pressure measurement unit for measuring an ambient atmospheric pressure, wherein
the arithmetic logic unit calculates the
information on the measurement accuracy based on the ambient atmos pheric pressure.

3.-5. (canceled)

6. A measurement method comprising:
in measuring biological information when a test site is in contact with a contact u nit,
a step in which a biological sensor acquires a biometric output from the test site;
a step in which a controller measures the biological information based on the biometric output;
a step in which an arithmetic logic unit calculates information on measurement accuracy of the biological information; and
a step in which an information output unit outputs the biological information and the information on the measurement accuracy.
